(19) United States
(12) Patent Application Publication
(10) Pub. No.: US 2022/0151706 A1
(43) Pub. Date: May 19, 2022

(54) ENHANCED REALITY MEDICAL GUIDANCE SYSTEMS AND METHODS OF USE

(71) Applicant: PETAL SURGICAL, INC., Foster City, CA (US)

(72) Inventors: Prashant CHOPRA, Foster City, CA (US); Salil S. JOSHI, Germantown, TN (US)

(73) Assignee: PETAL SURGICAL, INC., Foster City, CA (US)

(21) Appl. No.: 17/440,258

(22) PCT Filed: Mar. 23, 2020
(86) PCT No.: PCT/US2020/024212

§ 371 Date: Sep. 17, 2021

Related U.S. Application Data
(60) Provisional application No. 62/821,927, filed on Mar. 21, 2019.

(51) Int. Cl. A61B 34/20; A61B 6/12; A61B 6/00; A61B 90/00; G02B 27/01

(52) U.S. Cl. CPC A61B 34/20; A61B 6/12; A61B 6/4441; A61B 90/37; G02B 27/0172; G02B 2027/014; A61B 2034/2051; A61B 2034/2063; A61B 2034/2065; A61B 2090/365; G02B 2027/0138; A61B 2034/2057

(57) ABSTRACT

A system for displaying enhanced reality images of a body includes a fiducial marker patch, an external medical imaging system, a camera, a tool, a controller, and a display. The fiducial marker is configured to be placed on the body. The tool is configured to be inserted into the body for a medical procedure. The controller is configured to develop 2D or 3D images of the tool positioned within the body in real time based upon images from the external medical imaging system and the camera. The display is configured to display the 2D or 3D images in real time.

### CROSS-REFERENCE TO RELATED APPLICATIONS

This application claims priority to U.S. Provisional Application No. 62/821,927, titled “Enhanced Reality Medical Guidance Systems and Methods of Use,” filed Mar. 21, 2019, the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Augmented reality (AR) can generally be thought of as computer images overlaid on top of real images with the computer-generated overlay images being clearly and easily distinguishable from the real-world image. Healthcare applications are beginning to see a rise in the interest in use of augmented reality (AR) technologies to improve medical procedures, clinical outcomes, and long term patient care. However, due to certain fundamental challenges that limit the accuracy and usability of AR in life critical situations, the use of AR is yet to realize its complete potential in healthcare space. Accordingly, an improved AR system for the healthcare, and particularly the medical guidance space, is desired.

### SUMMARY OF THE DISCLOSURE

Described herein are devices, systems, and methods for combining various kinds of medical data to produce a new visual reality for a surgeon or health care provider. The new visual reality provides a user with the normal vision of the user's immediate surroundings accurately combined with a virtual three-dimensional model of the operative space and tools, enabling a user to “see through” the patient's body.

In some embodiments, a portable holographic endovascular guidance system is described. In other embodiments, a holographic endovascular guidance system integrated with an external imaging system, such as a fluoroscopy system, is described.

In general, in one embodiment, a system for displaying enhanced reality images of a body includes a fiducial marker patch, an external medical imaging system, a camera, a tool, a controller, and a display. The fiducial marker is configured to be placed on the body. The tool is configured to be inserted into the body for a medical procedure. The controller is configured to develop 2D or 3D images of the tool positioned within the body in real time based upon images from the external medical imaging system and the camera. The display is configured to display the 2D or 3D images in real time.

This and other embodiments can include one or more of the following features. The camera can be mounted on the external medical imaging system. The camera can be a visible light camera. The camera can be wearable. The external medical imaging system can be an x-ray system. The x-ray system can be a C-arm x-ray system. The external medical imaging system can be an ultrasound system. The external imaging system can be a drapeable or wearable imaging system. The tool may not include an imaging sensor thereon or therein. The patch can include radiopaque features. The patch can include infrared-visible features. The patch can include electromagnetic-wave-emitting features.

In general, in one embodiment, a method of displaying enhanced reality images of a body includes: (1) inserting a tool into the body for a medical procedure where the body includes a fiducial marker patch thereon; (2) imaging the fiducial marker on the body with a camera, (3) imaging the fiducial marker on the body with an external medical imaging system, (4) developing 2D or 3D images of the tool inserted into the body in real time based upon images from the external medical imaging system and the camera, and (5) displaying the 2D or 3D images.

This and other embodiments can include one or more of the following features. The method can further includes estimating a 3D position and 3D orientation of the patch based upon images of the patch from the external medical imaging system. The patch can include at least one radiopaque feature. Estimating can include detecting a 2D projection of the radiopaque feature in the images from the external medical imaging system. The method can further include estimating a 3D position and 3D orientation of the patch based upon visual features in images of the patch from the camera. Estimating can include detecting a 2D project of radiopaque features of the patch. Estimating can include estimating a real 3D pose of the patch based upon a geometry of the patch. The method can further include comparing pre-acquired images to images from the external medical imaging system and the camera. The method can further include estimating a transform between the pre-acquired images, images from the external medical imaging system, and images from the camera. The method can further include deforming the pre-acquired images based upon the comparison.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a schematic of a holographic endovascular guidance system.

FIGS. 2A-2D show a holographic endovascular guidance system.

FIGS. 3A-3B show exemplary displays of a dynamic vascular map.

FIGS. 4A-4C show a patch for use with a holographic endovascular guidance system.

FIGS. 5A-5B show use of a holographic endovascular guidance system to display multiple different endovascular views.

FIG. 6 shows a holographic endovascular guidance system wherein the interventional tool does not include a sensor thereon.

FIGS. 7A-7C show exemplary holographic displays from a holographic endovascular guidance system.

FIG. 8 shows a holographic endovascular guidance system wherein the interventional tool includes a sensor thereon.

FIGS. 9A-9B show various view of a holographic endovascular guidance system.

FIGS. 10A-10C show the use of a holographic endovascular guidance system to cross a vascular stenosis or occlusion with two or more tools.

### DETAILED DESCRIPTION

Described herein are systems for the 3D display of images, such as for medical guidance. For example, a portable holographic endovascular guidance system is described herein.

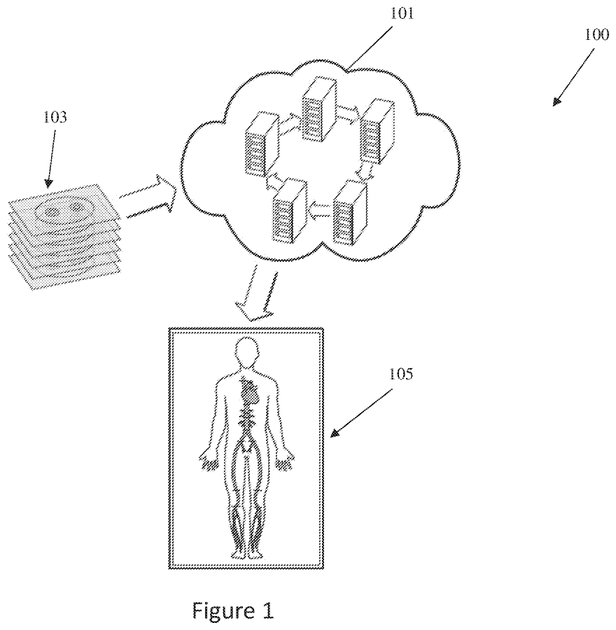

Referring to FIG. 1, in some embodiments, a portable holographic endovascular guidance system 100 can include an artificial intelligence powered “deformable” vascular map extraction subsystem. The system 100 thus includes a computing network 101 (e.g., local/cloud/network). Pre-operative diagnostic images 103 (e.g., CT scan images) can be input into the network 101. A resulting image 105 can be processed by: (1) extracting a vascular or organ mask (binary or probabilistic); (2) identifying deformable units and the linkages between them; (3) refining the deformable units and their relationship tree using a dynamic deep learning computing network that utilizes prior knowledge of real human images; and/or (4) estimating physical and functional characteristics of the vascular system at one or more locations on the map, such as the nature of blockages, the size of blockages, the age of the vessel, the plasticity of the vessel, the flow rate of blood in the vessels, or a treatment plan for a particular disease site. Such a treatment plan can include, for example, whether to perform surgery or catheter intervention (e.g., whether to use a stent or balloon and/or perform shaving or drug delivery) and/or the steps for recommended treatment (e.g., incision sites, size of incision, position/orientation of approach to the site, size/kind of tools to use, and/or path to approach the site).

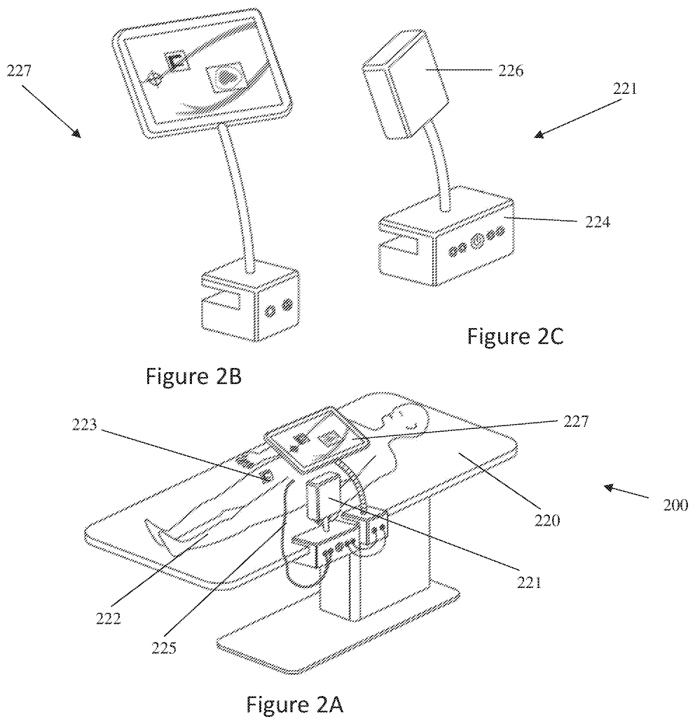

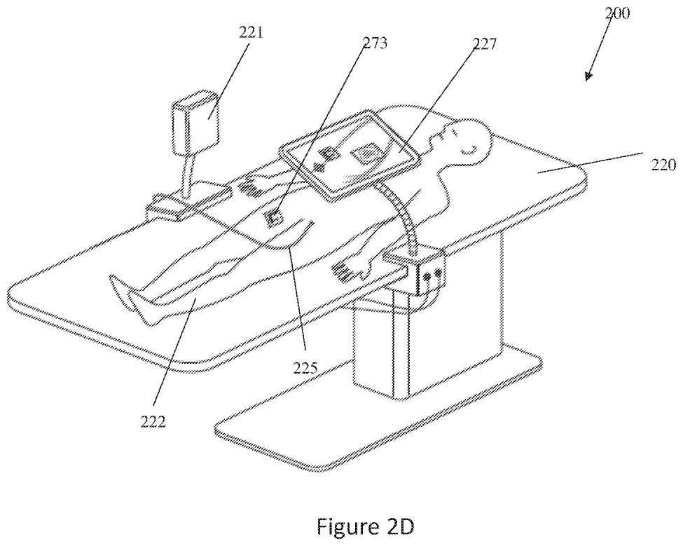

Referring to FIGS. 2A-2D, in some embodiments, a portable holographic endovascular guidance system 200 can include a sensing system 221, a patient patch 223, one or more sensed tools 225, and a display mechanism 227. The sensing system 221 includes a base 224 configured to attach to a table 220 (e.g., with a clamping mechanism). The base 224 can include a processor therein, a power switch, and two or more connection sockets. The sensing system 221 further includes a field sensor 226, such as an electromagnetic field generator. The one or more sensed tools 225 can include a main conduit to accept a medical tool (e.g., a guidewire, catheter, camera, or an elongate platform that includes a single energy source for visualization of obstruction and re-canalization), a sensor conduit with one or more sensors embedded in it, sensing features (visual, infrared, or ultra-violet), and a connector on the proximal end to connect to the main sensing system 221 and/or to an energy/imaging system (when an elongate platform with a single energy source is used). The sensing features can be unique to the system 200 and thus decipherable only by the system 200. The display mechanism 227 can serve as the main visualization display. In some embodiments, the display mechanism 227 can be a tablet that includes a built-in camera (and/or the camera can be attached to the display mechanism 227). The camera can be, for example, a visible light or infrared modality camera configured to point at the patient 222. The display mechanism 227 can include a processor therein as well as a display panel (e.g., that is flat and/or that provides a natural holographic display). In some embodiments, the display mechanism 227 can further include a camera pointed at the user (e.g., the physician), which can be useful for gesture control (e.g., for when the physician is scrubbed and cannot touch equipment), to monitor scene lighting conditions to dynamically tune the marker detection algorithms, to model the procedure room (e.g., 3D from 2D video), and/or to gather information on physician skills for user experience improvement. The patient patch 223 can include one or more sensing features (e.g., visual, infrared, or ultra-violet) that are unique to system 200 and can be deciphered only by system 200.

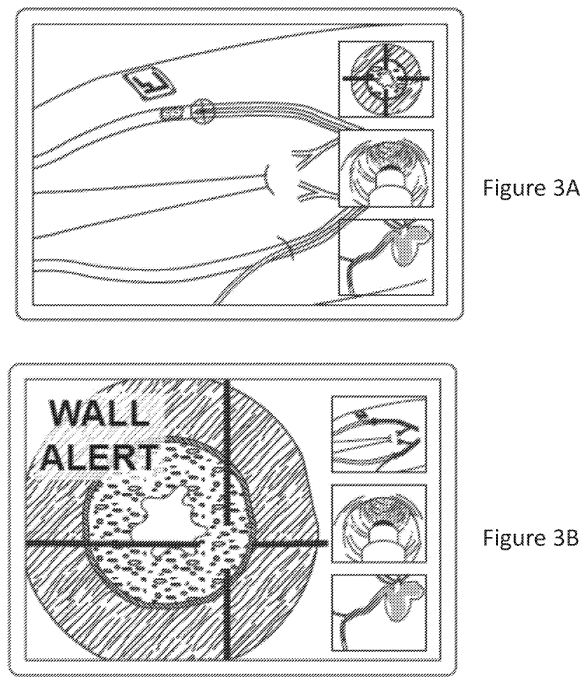

In use of the system 200, the processor of the display mechanism 227 (and/or a separate processor of system 200 and network/cloud component) can be configured to: (1) estimate the 3D position and orientation of the patient patch 223 using the embedded sensor's readings in the system system's space; (2) estimate the 3D position and orientation of the patient patch 223 using the visual features in the holographic display's face; (3) estimate the 3D position and orientation of one or more of the sensed tools 225 using the embedded sensors' readings in the sensing system's space; (4) estimate a transform between the sensing system 221, the pre-operative images' system, and the holographic display system; (5) estimate the best position and orientation of the patient patch 223 in all spaces it is visible in; (6) estimate the best position and orientation of the sensed tools 225 in all spaces it is visible in; (7) deform the vascular map from pre-operative images to match the best estimate of step 6; and/or (8) display a dynamically deforming context containing the sensed tool 225 and the vascular map and other live sensed information on the holographic display 227 in near-real-time. Exemplary displays of the dynamically displayed map are shown in FIGS. 3A and 3B.

Additionally, the processor of system 200 can be configured to estimate the physical and functional characteristics of the vascular system during or after treatment in the patient body at a corresponding map location using live sensors (e.g., on the sensing tools 225 or on an external sensor, such as a leg or thigh wrap), or via analysis of images acquired live using external or internal imaging systems. Such characteristics can include the nature of the blockages, the size/shape of blockages, the age of the vessel, the plasticity of the vessel, the flow rate of blood in the vessels, and/or the ideal treatment plan for the residual vessel disease at specific sites (such as whether to perform surgery or catheter intervention and/or steps for a follow up treatment). In some embodiments, the processor of system 200 can further be configured to present a comparison of the determined/estimated physical and functional characteristics of the vascular system before and after treatment to assess the success of the treatment against the prescription.

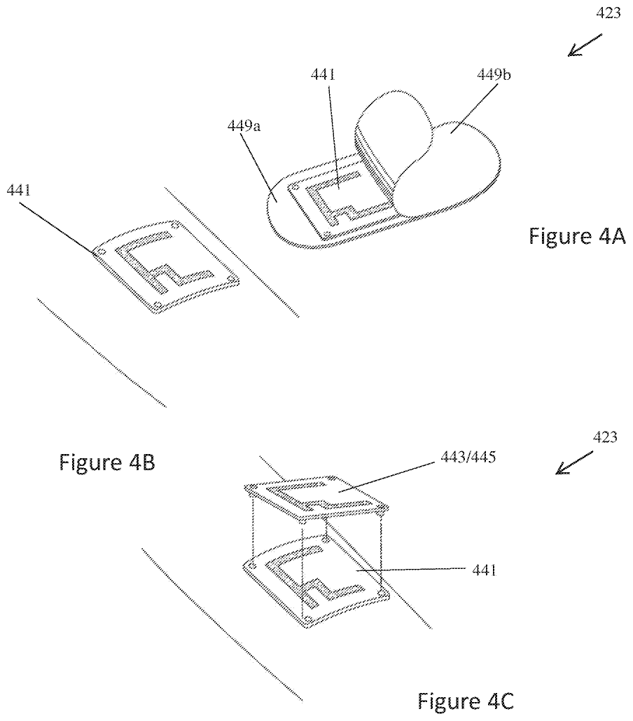

Referring to FIGS. 4A-4C, in some embodiments, a patch 423 can include multiple layers. The base layer 441 can be flexible and include an adhesive layer for adhering to the skin, similar to a band-aid. The base layer 441 can be visible in the diagnostic images taken prior to vascular map extraction and can include physical, electromagnetic, gluing, or mechanical features to accept a middle layer 443 in exactly/only one orientation. The middle layer 443 can also be flexible, but can include enough thickness (e.g., 1-10 mm) to allow embedding of sensors or emitters (e.g., electromagnetic or radiopaque or radio wave) in a precise pattern. A connector in the middle layer 443 can be configured to connect to the main sensing system (e.g., sensing system 221). The top layer 445 can be configured to sit in a precise orientation relative to the middle layer 443 and can include sensing features (visual, infra-red, or ultra-violet) that are unique and/or decipherable only by the system. The features can be static (i.e., one-time use) or on a programmable electronic/electrical display (reusable). As shown in FIG. 4A, the patch 423 can be stored between two disposable covers 449a,b.

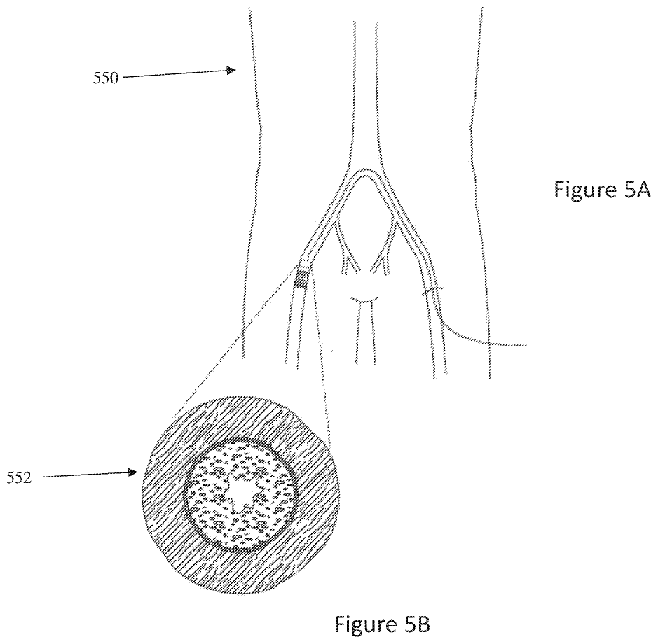

Referring to FIGS. 5A-5B, in some embodiments, a portable holographic endovascular guidance system as described herein can detect a partial lesion or partial blockage in the right iliac artery and show a 3D holograph 550 and/or cross-sectional view 552.

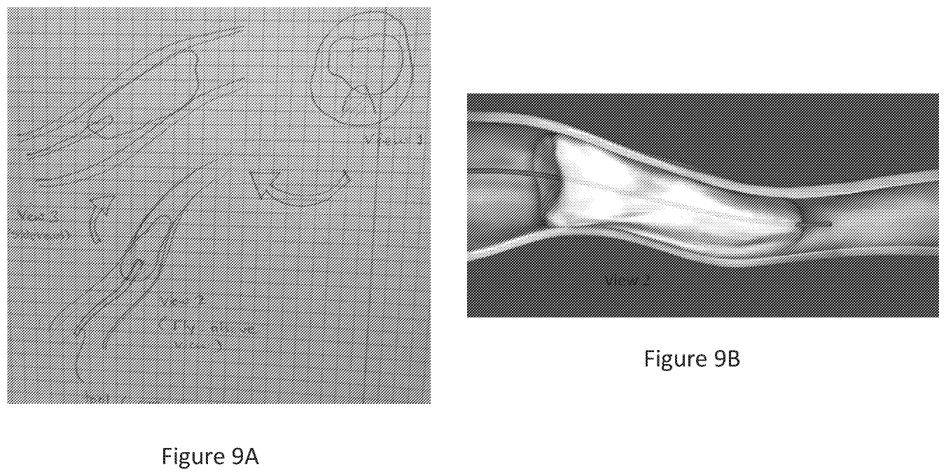

Referring to FIGS. 9A-9B, in some embodiments, a portable holographic endovascular guidance system as described herein can display a set of different endovascular views. These different views (e.g., three views) can show the patent vessel proximal to a blockage, the blockage itself, and patent vessel distal to the blockage, all in the same demonstration.

A holographic endovascular guidance system integrated with an external imaging system, such as a fluoroscopy system, is also described herein.

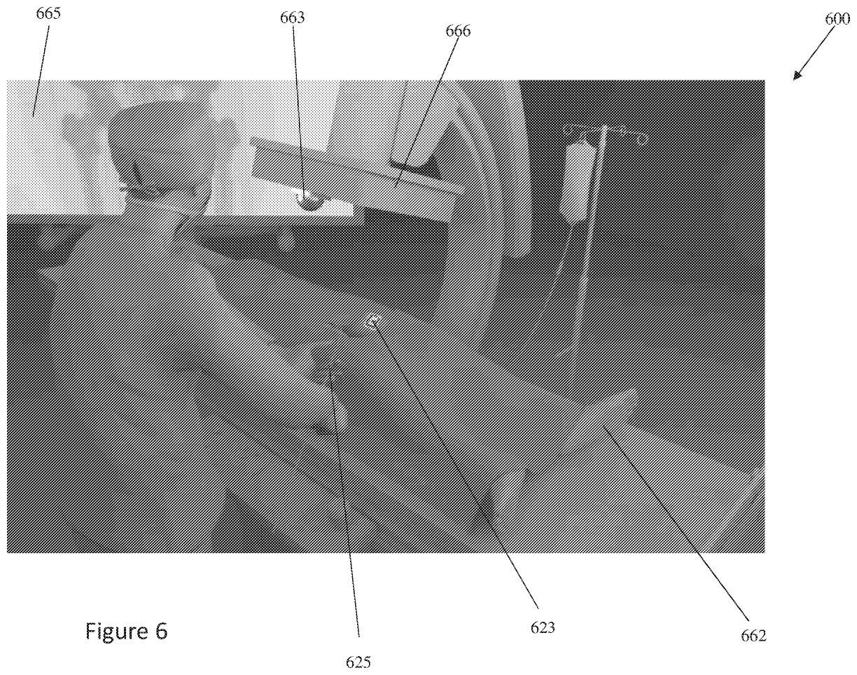

As described above with respect to systems 100 and 200, the holographic endovascular guidance system integrated with an external imaging system can include an artificial intelligence powered “deformable” vascular map extraction subsystem. Additionally, as shown in FIG. 6, a system 600 comprising holographic endovascular guidance system integrated with an external imaging system 666 can include a patch 623 (e.g., similar to patch 223 or patch 423), one or more flexible tools 625, a camera system 663 (e.g., mounted to an external imaging system 666), and a display mechanism 665. The flexible tools 625 can be similar to flexible tools 225 except that the tools may not include sensors thereon. The external imaging system 666 can be, for example, a C-arm x-ray or ultrasound (or in some embodiments, it can be a patient drapeable or wearable vest-based imaging system). In some embodiments, the camera system 663 can include a visible light or infrared camera configured to view the patient 622 and the patch 623. The camera system 663 can be wired or wirelessly connected to the processor 661. The display mechanism 665 can be a flat panel or a natural holographic display.

The system 600 can further include a processor. The processor can include software or firmware locally or on a networked cloud component that is configured to: (1) estimate the 6D pose (3D position and 3D orientation) of the patient patch 623 using the embedded sensors' readings in the x-ray system's space, including detecting the 2D projection of the patch's radiopaque features in an x-ray image; (2) estimate the 3D position and orientation of the patient patch 623 using the visual features in the enhanced reality camera's space, including detecting the 2D projection of the patch's radiopaque features in a camera image and/or estimating the real 3D pose of the patch in camera's 3D space using patch's geometry; (3) estimate the position and orientation of the tool 625 inside the patient's body using the external imaging system 666 in the respective imaging system's space; (4) estimate a transform between the pre-operative images' system, the external imaging system 666, and the holographic display system; (5) estimate the best position and orientation of the patient patch 623 in all spaces it is visible in; (6) estimate the best position and orientation of the tool 625 in all spaces it is visible in; (7) deform the vascular map from pre operative images to match the best estimate found in step 6; and/or (8) blend the x-ray and 3D images and display a dynamically deforming context containing the tool and the vascular map and other live sensed information on the holographic display in near-real-time.

Blending the x-ray and 3D images can include: (A1) registering the detected 2D feature points of the patch in the x-ray image with detected 2D feature points in the camera's space; and (A2) carrying the 6D pose of the patch in the camera's space to the x-ray imaging system through the 2D registration transform; OR (B1) extracting the 6D pose of the patient patch solely based on its known geometry and the characteristics of the x-ray imaging system; and (B2) matching the patch's pose estimate with the one estimated by the ‘real’ camera to localize it in both frames of references with double the accuracy. The blending can further include (3) using the result to generate a new 3D overlay image of the deforming vascular map upon every change of the x-ray imaging system's orientation. To dynamically adjust the 3D model, the 3D model can be constantly deformed such that both the features of the patch 623 match and the specific areas (e.g., branches of a vascular system) match. For example, because each of the branches of a vessel has limited degrees of freedom, those branches can be used as hinge points that can be dynamically moved in the 3D overlay as the x-ray view/orientation changes. The 3D overlay can thus be produced because the pose of the patch 623 is known in the camera's space, and the pose of the x-ray system can be estimated based on a match of the x-ray and the real camera image.

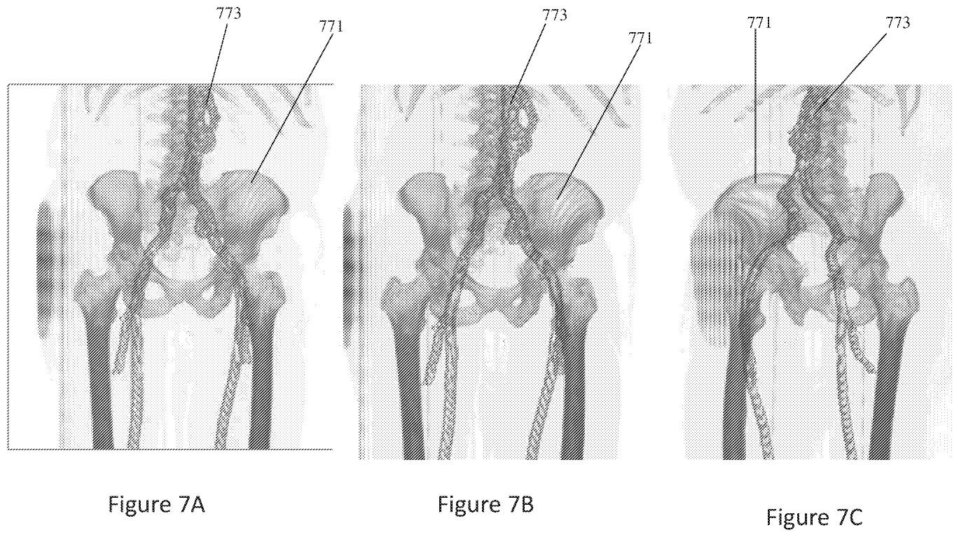

FIGS. 7A-7C show exemplary resulting holographic displays when a 3D overlay image 773 is placed over an x-ray image 771. As shown, as the orientation of the camera is changed (e.g., as the angle of the x-ray image changes), the vessels in the 3D overlay can deform to compensate.

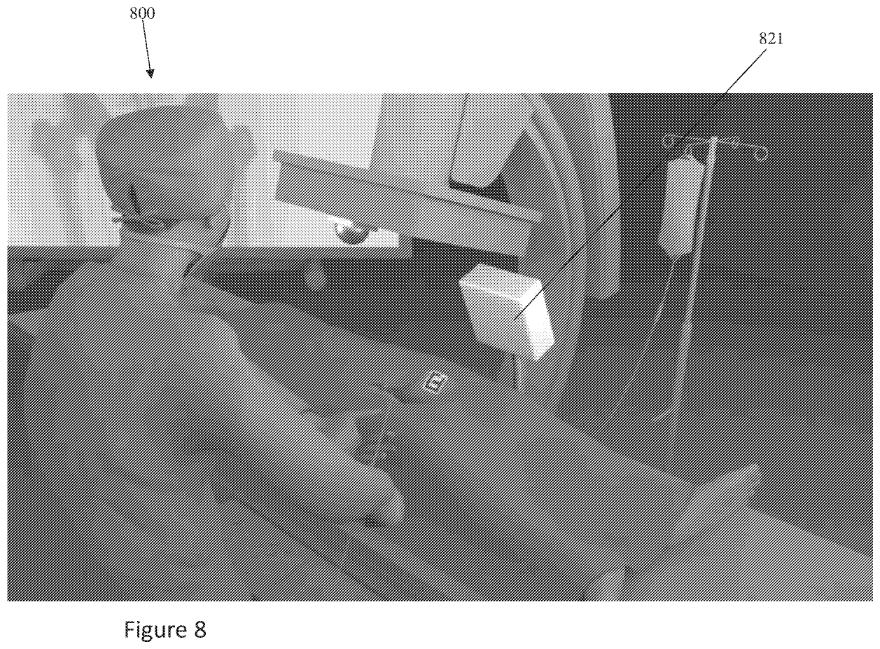

It should be understood that while not described as being used with a sensed tool, system 600 can also be used with a sensed tool. The sensed tool can provide additional details for create of the 3D image over the x-ray image. Such a system 800 is shown in FIG. 8. The system 800 is similar to system 600 except that it additionally includes a sensing system 821 configured to sense the tool.

In embodiments where a sensor on the tool is used to create the 3D model, an endovascular probe can alternatively be used in place of a sensing system and camera.

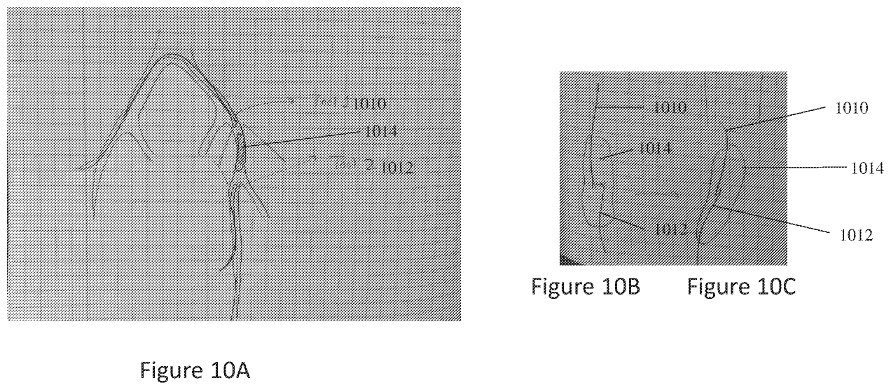

In some embodiments, the systems described herein can be used to help cross vascular stenoses or occlusions using two or more tools (e.g., sensed or unsensed tools). Referring to FIG. 10A, the first tool 1010 and the second tool 1012 can approach the same lesion 1014 from opposite directions. Using the systems described herein, the relative poses of the first tool 1010 and the second tool 1012 can be known throughout the procedure. Once in proximity to each other (as shown in FIG. 10B), the first tool 1010 and the second tool 1012 can snap together in a predetermined configuration (as shown in FIG. 10C). For example, a magnetic system can be used to bond the tools 1010, 1012 together in a single unit. Once attached, the tools 1010, 1012 may be withdrawn in either preferred direction (either antegrade or retrograde), making an artificial conduit through the vascular occlusion, thereby achieving recanalization. In some embodiments, one or more of the tools 1010, 1012 can include an energy source, such as a laser, to aid in moving through the lesion 1014.

Additional holographic systems for medical guidance are described in International Application No. PCT/US2017/054868, filed Oct. 3, 2017, the entirety of which is incorporated by reference herein. The features of the systems described herein can be combined with or substituted for any of the features of the systems described in International Application No. PCT/US2017/054868, filed Oct. 3, 2017.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for displaying enhanced reality images of a body, the system comprising:
a fiducial marker patch configured to be placed on the body;
an external medical imaging system;
a camera;
a tool configured to be inserted into the body for a medical procedure;
a controller configured to develop 2D or 3D images of the tool positioned within the body in real time based upon images from the external medical imaging system and the camera; and
a display configured to display the 2D or 3D images in real time.

2. The system of claim 1, wherein the camera is mounted on the external medical imaging system.

3. The system of claim 1, wherein the camera is a visible light camera.

4. The system of claim 1, wherein the camera is wearable.

5. The system of claim 1, wherein the external medical imaging system is an x-ray system.

6. The system of claim 5, wherein the x-ray system is a C-arm x-ray system.

7. The system of claim 1, wherein the external medical imaging system is an ultrasound system.

8. The system of claim 1, wherein the external imaging system is a drapeable or wearable imaging system.

9. The system of claim 1, wherein the tool does not include an imaging sensor thereon or therein.

10. The system of claim 1, wherein the patch comprises radiopaque features.

11. The system of claim 1, wherein the patch comprises infrared-visible features.

12. The system of claim 1, wherein the patch comprises electromagnetic-wave-emitting features.

13. A method of displaying enhanced reality images of a body, comprising:
inserting a tool into the body for a medical procedure, the body including a fiducial marker patch thereon;
imaging the fiducial marker on the body with a camera;
imaging the fiducial marker on the body with an external medical imaging system;
developing 2D or 3D images of the tool inserted into the body in real time based upon images from the external medical imaging system and the camera; and
displaying the 2D or 3D images.

14. The method of claim 13, further comprising estimating a 3D position and 3D orientation of the patch based upon images of the patch from the external medical imaging system, wherein the patch includes at least one radiopaque feature.

15. The method of claim 14, wherein estimating comprises detecting a 2D projection of the radiopaque feature in the images from the external medical imaging system.

16. The method of claim 13, further comprising estimating a 3D position and 3D orientation of the patch based upon visual features in images of the patch from the camera.

17. The method of claim 16, wherein estimating comprises detecting a 2D project of radiopaque features of the patch.

18. The method of claim 16, wherein estimating comprises estimating a real 3D pose of the patch based upon a geometry of the patch.

19. The method of claim 13, further comprising comparing pre-acquired images to images from the external medical imaging system and the camera.

20. The method of claim 19, further comprising estimating a transform between the pre-acquired images, images from the external medical imaging system, and images from the camera.

21. The method of claim 19, further comprising deforming the pre-acquired images based upon the comparison.
